(19) United States
(12) Patent Application Publication
(10) Pub. No.: US 2021/0282663 A1
(43) Pub. Date: Sep. 16, 2021

(54) CAPNOGRAPHY WITH LEAD SELENIDE DETECTOR AND INTEGRATED BANDPASS FILTER

(71) Applicant: KONINKLIJKE PHILIPS N.V., EINDHOVEN (NL)

(72) Inventors: ZHI-XING JIANG, SOUTHBURY, CT (US); SZILVESZTER CSEH JANDO, NAUGATUCK, CT (US)

(21) Appl. No.: 16/478,031

(22) PCT Filed: Jan. 9, 2018
(86) PCT No.: PCT/EP2018/050386

§ 371 Date: Jul. 15, 2019

Related U.S. Application Data
(60) Provisional application No. 62/446,622, filed on Jan. 16, 2017.

(51) Int. Cl. A61B 5/08; G01N 21/3504

(52) U.S. Cl. CPC A61B 5/082; A61B 2560/0252; G01N 21/3504

(57) ABSTRACT

A capnometer (10) includes an integrated device (30, 30A, 30B) comprising a substrate (40), a lead selenide (PbSe) layer (42) or other infrared light absorbing layer disposed on the substrate, and a bandpass filter layer (50) disposed on the substrate. A temperature sensor tracks temperature of the device. A CO₂ measurement cell (20) has light source (28) arranged to emit light (L) that passes through a flow path (F) and then through the bandpass filter layer of to reach the PbSe or other infrared light absorbing layer. Electronics (32) connected with the PbSe or other infrared light absorbing layer measure a photoconductivity signal. The electronics includes signal processing circuitry to convert the photoconductivity signal to a carbon dioxide partial pressure or concentration value. The electronics also includes the temperature sensor to enable accurate and fast temperature control of the device and instant temperature compensation for the temperature change.

### FIELD

The following relates generally to the capnography arts, medical monitoring arts, infrared detector arts, and related arts.

### BACKGROUND

A capnometer measures the concentration or partial pressure of carbon dioxide (CO₂) in respiratory gases. The CO₂ waveform can provide information such as end-tidal carbon dioxide (etCO₂) which is a useful vital sign in assessing patients with respiratory problems, for assessing efficacy of mechanical respiration, and so forth.

In a typical capnometer design, a respired gas flow is accessed in either a mainstream configuration in which a CO₂ measurement cell is in-line with the respiratory circuit (e.g. in the mechanical ventilator airflow circuit) or in a sidestream configuration in which respired gas is drawn off the main flow using a pump. Infrared light is transmitted through respired gas flow. CO₂ absorbs significantly in the infrared, with an absorption peak at about 4.26 micron. In a typical optical detection setup, a bandpass filter having a pass band in the 3-5.5 micron range is used to isolate the CO₂-sensitive infrared spectral range, and a lead selenide (PbSe) detector made of a thin film of lead selenide deposited on a quartz substrate is used to detect the transmitted infrared light intensity. The PbSe film exhibits photoconductivity for infrared light in the wavelength range of 3 to 5.5 microns. As both the photoconductivity of the PbSe layer and the central wavelength of the pass band of the bandpass filter can vary with temperature, temperatures of both the PbSe detector and the bandpass filter typically are accurately monitored by thermocouples or other temperature sensors. These sensors provide feedback control for maintaining PbSe detector and the filter at the designed operating temperature or compensation of the ambient temperature change through special calibration and algorithm.

The following discloses a new and improved systems and methods that address the above referenced issues, and others.

### SUMMARY

In one disclosed aspect, a capnometer is disclosed. An integrated device comprises a substrate, a lead selenide (PbSe) layer or other infrared light absorbing layer disposed on the substrate, and a bandpass filter layer disposed on the substrate. A light source is arranged to emit light that passes through the bandpass filter layer of the integrated device to reach the PbSe or other infrared light absorbing layer of the integrated device. Electronics are connected with the PbSe or other infrared light absorbing layer of the integrated device to measure a photoconductivity signal of the PbSe or other infrared light absorbing layer. The electronics include signal processing circuitry to convert the photoconductivity signal to a carbon dioxide partial pressure or concentration value.

In another disclosed aspect, an infrared light detector comprises: a substrate; a lead selenide (PbSe) layer disposed on the substrate; electrodes disposed on the substrate and electrically connected with the PbSe layer; and a bandpass filter layer disposed on the substrate, the bandpass filter layer having a pass band encompassing 4.26 micron.

One advantage resides in providing a more compact infrared light detector.

Another advantage resides in providing a more compact capnometer.

Another advantage resides in providing a capnometer with reduced components (i.e. fewer parts).

Another advantage resides in providing an infrared light detector with reduced reflection loss.

Another advantage resides in providing a capnometer with improved sensitivity.

Another advantage resides in providing a more accurate, consistent and correlated measurement of temperatures of both IR sensing element (PbSe) and IR selection element (narrow bandpass filter) with improved temperature control and/or compensation of ambient temperature change.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a capnometer.

FIG. 2 diagrammatically illustrates a side sectional view of the integrated PbSe detector and bandpass filter component of the capnometer of FIG. 1.

FIGS. 3 and 4 diagrammatically illustrate side sectional views of alternative embodiments of the integrated PbSe detector and bandpass filter component of the capnometer of FIG. 1.

### DETAILED DESCRIPTION

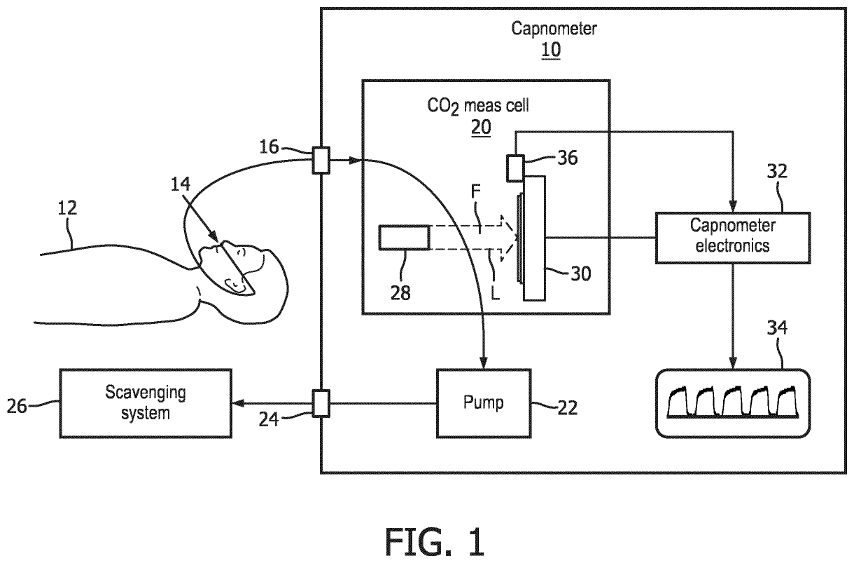

With reference to FIG. 1, an illustrative capnometer 10 is connected with a patient 12 by a suitable patient accessory, such as a nasal cannula 14 in the illustrative example, or by an airway adaptor connecting with an endotracheal tube used for mechanical ventilation, or so forth. The patient accessory 14 may optionally include one or more ancillary components, such as an air filter, water trap, or the like (not shown). In the illustrative capnometer 10, respired air is drawn from the patient accessory 14 into a capnograph air inlet 16 and through a carbon dioxide (CO₂) measurement cell 20 by an air pump 22. The air is then discharged via an air outlet 24 of the capnometer 10 to atmosphere or, as in the illustrative embodiment, is discharged through the air outlet 24 into a scavenging system 26 to remove an inhaled anesthetic or other inhaled medicinal agent before discharge into the atmosphere.

The illustrative capnometer setup has a sidestream configuration in which respired air is drawn into the capnometer 10 using the pump 22, and the CO₂ measurement cell 20 is located inside the capnometer 10. The sidestream configuration is suitably used for a spontaneously breathing patient, i.e. a patient who is breathing on his or her own without assistance of a mechanical ventilator. In an alternative configuration, known as a mainstream configuration (not illustrated), the CO₂ measurement cell is located externally from the capnometer device housing, typically as a CO₂ measurement cell patient accessory that is inserted into the “mainstream” airway flow of the patient. Such a mainstream configuration may, for example, be employed in conjunction with a mechanically ventilated patient in which the CO₂ measurement cell patient accessory is designed to mate into an accessory receptacle of the ventilator unit, or is installed on an airway hose feeding into the ventilator.

The CO₂ measurement cell 20 comprises an infrared optical absorption cell in which carbon dioxide in the respired air drawn from the patient accessory 14 produces absorption in the infrared that is detected optically. CO₂ has an absorption peak at about 4.26 micron, and in some embodiments measurements are done within the 3-5.5 micron range inclusive or some sub-range of that range (preferably including 4.26 micron). To this end, a light source 28 emits light L over a spectrum that encompasses the desired measurement band (e.g. 3-5.5 micron or some sub-range thereof, preferably including 4.26 micron). The light L may extend spectrally beyond the detection spectral range, because it will be filtered as described below. In some embodiments the light source 28 may include an optical chopper, pulsed power supply, or the like in order to deliver the emitted light L as light pulses. The emitted light L transmits through a flow path F along which the respired gas flows. The flow path F may be defined by a tube or other conduit defining a cuvette with walls made of a plastic, glass, sapphire, or other material that is transparent over the range of interest (e.g. over 3-5.5 micron). In the sidestream arrangement, the pump 22 actively drives the flow of respired gas through the flow path F; in a mainstream configuration the flow may be driven by mechanical ventilation of the patient, and/or by active breathing of the patient.

An integrated device 30 including both a light detector layer and a bandpass filter operates to both filter light L so as to pass light in a pass band, e.g. 3-5.5 micron or some sub-range thereof (preferably including 4.26 micron) and to detect the light that passes the bandpass filter, e.g. using a lead selenide (PbSe) layer. Capnometer electronics 32 provide electrical biasing of the detector layer of the integrated device 30 and measure a detector signal (e.g. a voltage, current, or resistance) from the detector layer. For example, the electronics 32 may drive a fixed electric current through the PbSe layer and measure the voltage so as to output a voltage signal (or, alternatively, a resistance signal computed as V/I where V is the measured voltage and I is the applied current). Alternatively, the electronics 32 may apply a fixed voltage over the PbSe layer and measure the current so as to output a current signal (or, alternatively, a resistance signal computed as V/I where V is the applied voltage and I is the measured current). The applied and measured signals may in general be d.c. or a.c. or some combination (e.g. an a.c. signal superimposed on a d.c. bias). The electronics 32 also optionally include analog signal processing circuitry and/or digital signal processing (DSP) suitable for converting the detected signal into a capnography signal, e.g. a concentration or partial pressure of CO₂ in the respired gas flow, and optionally for performing further processing such as detecting a breath interval and/or an end-tidal CO₂ level (etCO₂ level). The conversion to CO₂ level can employ suitable empirical calibration—in general, higher CO₂ concentration or partial pressure in the flow F produces greater absorption and a reduced capnography signal voltage. The empirical calibration may take into account other factors such as flow rate or pressure, and/or the effects of other gases such as oxygen and nitrous oxide which can affect the infrared absorption characteristics, as is known in the art, and can be suitably programmed as a look-up table, mathematical equation, non-linear op-amp circuit, or so forth. In the case of the capnometer electronics 32 being implemented at least in part by DSP, such DSP may be implemented by a microcontroller or microprocessor or the like programmed by instructions stored on a read only memory (ROM), electronically programmable read-only memory (EPROM), CMOS memory, flash memory, or other electronic, magnetic, optical or other non-transitory storage medium that is readable and executable by the microcontroller or microprocessor or the like to perform the digital signal processing. For DSP processing, a front-end analog-to-digital (A/D) conversion circuit is typically provided to digitize the detector signal. An output component 34 is provided to output the capnometer signal or digital data generated by the capnometer electronics 32. The illustrative output component is a display 34, e.g. an LCD display or the like. The illustrative display 34 plots CO₂ concentration or partial pressure versus time as a trendline. Additionally or alternatively, the display may show a numerical value, e.g. of the etCO₂. The output component may additionally or alternatively take other forms, such as being or including (possibly in addition to the display 34) a USB port or other data port via which the capnometer data may be read out. Moreover, the capnometer electronics 32 may perform additional functions such as monitoring a thermocouple, temperature diode, or other temperature sensor 36 that measures the operating temperature of the integrated device 30. This is useful because a large change in temperature of the integrated device 30 can produce an undesired shift in the pass band of the bandpass filter and/or in the detector sensitivity. In some embodiments, the integrated device 30 may be mounted on or in thermal contact with a Peltier device or other thermoelectric cooling device (not shown) and the electronics 32 operates the thermoelectric cooling device in a feedback control mode using the temperature from the temperature sensor 36 to maintain the integrated device 30 at a design-basis operating temperature. It will be further appreciated that the capnometer 10 may include numerous other components not illustrated in simplified diagrammatic FIG. 1, such as a pressure gauge and/or flow meter for monitoring the respired gas flow, a keypad or other user input components, and/or so forth.

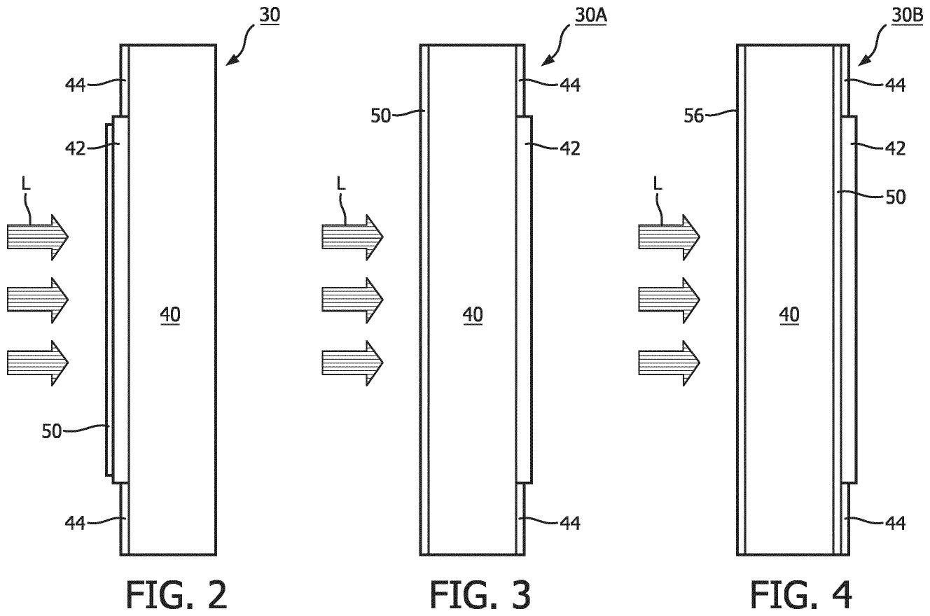

With reference now to FIG. 2, a first embodiment of the integrated device 30 is shown. In this embodiment, the integrated device includes a substrate 40, such as a quartz substrate by way of non-limiting illustrative example. The substrate 40 is preferably planar, e.g. a wafer or relatively thin chip having two flat opposing principal sides. A lead selenide (PbSe) layer 42 is disposed on one side of the substrate 40. The PbSe layer 42 is sufficiently thick to absorb most light in the target range, e.g. 3-5.5 micron in some embodiments, or a smaller spectral range encompassing 4.26 micron in other embodiments. In some illustrative embodiments the PbSe layer 42 has a thickness between 0.5 micron and 2.5 micron inclusive. The PbSe layer 42 may, for example, be deposited by chemical bath deposition (CBD) or vapor phase deposition (VPD). Typically, CBD is preferable due to faster readily achievable deposition rates. The deposited PbSe layer 42 is usually a polycrystalline film. Electrodes 44 are also disposed on the substrate 40 and are electrically connected with the PbSe layer 42 to enable measurement of a photoconductivity signal of the PbSe layer 42, e.g. a photoconductivity signal consisting of one of resistance, current, and voltage across the PbSe layer 42. The electrodes 44 may, for example, be gold or silver pads disposed over ends of the PbSe layer 42 or disposed directly on the substrate 40 adjacent the ends of the PbSe layer 42. The electrodes 44 may optionally include an adhesion layer and/or a diffusion barrier layer for promoting adhesion of the gold or silver pad layer to the PbSe and/or substrate material. The adhesion and/or diffusion barrier layer(s) may, for example, comprise one or more layers of chromium, titanium, titanium-tungsten, nickel, or so forth.

With continuing reference to FIG. 2, the integrated device 30 further includes a bandpass filter layer 50, which in the embodiment of FIG. 2 is disposed on top of the PbSe layer 42. As shown in FIG. 2, the light L generated by the light source 28 (see FIG. 1) passes through the bandpass filter layer 50 to reach the PbSe layer 42; thus, photoconductivity induced in the PbSe layer 42 by the light L is limited to the spectral portion of light L lying in the pass band of the bandpass filter layer 50. The bandpass filter layer 50 may be a broadband filter comprising a single layer or a plurality of layers operating by bulk absorption and/or optical interference to define a relatively broad pass band, e.g. 3-5.5 micron passband in some embodiments. Alternatively, the bandpass filter layer 50 may be a narrowband filter having a narrow pass band. In this approach the bandpass filter layer 50 comprises a multi-layer stack defining an interference filter. Such an interference filter can be designed using ray tracing approaches or other optical filter design techniques to have a narrow pass band tailored transitions between the pass band and the surrounding “stop” bands. Thus, for example, it is contemplated to design the bandpass filter layer 50 as a multi-layer stack defining an interference filter with a narrow pass band that encompasses a narrow transmission band at wavelength of 4.26 micron (the principal absorption line of CO₂) but does not encompass the absorption lines of other gases such as oxygen or nitrous oxide. Such an approach can simplify design of the signal processing circuitry (e.g. DSP or analog signal processing circuitry) of the electronics 32 (see FIG. 1) by reducing or eliminating the correction for these interfering gases when converting the photoconductivity signal measured for the PbSe layer 42 into a CO₂ concentration or partial pressure.

Disposing the bandpass filter layer 50 on top of the PbSe layer 42 provides an additional benefit, namely refractive index matching. PbSe has a refractive index of about n=4.9 for the wavelength of 4.26 micron. When the infrared light impinges on a bare PbSe surface, transmission of the infrared light into the PbSe layer is reduced significantly by reflection at the large refractive index step from air (n=1.0) to PbSe (n=4.9). By some estimates, 44% of the light is reflected at a bare PbSe surface, leaving only 56% of the light to penetrate into the PbSe. In the integrated device 30 of FIG. 2, by coating the PbSe layer 42 with the bandpass filter layer 50, the refractive index step is reduced thus reducing the optical reflection loss as compared with a bare PbSe surface. To reduce reflection loss, the bandpass filter layer 50 should comprise material with refractive index that is intermediate between n=1.0 of air and n=4.9 of PbSe. Some suitable materials include, by way of non-limiting example: Aluminum Oxide (Al₂O₃), Titanium Oxide (TiO₂), Cryolite, Magnesium Fluoride (MgF₂), Calcium Fluoride (CaF₂), Zinc Selenide (ZnSe), Zirconia (ZrO₂), Zinc Sulfide (ZnS), Spinel (MgAl₂O₄), Lithium Fluoride (LiF), Lead Fluoride (PbF₂), Cadmium Fluoride (CdF₂), Cadmium Sulfide (CdS), Zinc Oxide (ZnO), Silicon, Silicon Dioxide (SiO₂), Germanium, Germanium Oxide (GeO₂), Lead Telluride, Gallium Arsenide (GaAs), Thorium Fluoride, Hafnium Oxide, Tantalum Oxide, Niobium Oxide, Cadmium Sulfide, Antimony Fluoride, Yttrium Fluoride, or various multilayer combinations thereof (e.g. to define an interference filter).

One potential difficulty with the integrated device 30 of FIG. 2 is that the PbSe layer 42, e.g. deposited by CBD or CVD, is typically a polycrystalline layer having relatively high surface roughness. When the bandpass filter layer 50 is deposited on top of the PbSe layer 42, the resulting bandpass filter layer 50 may assume roughness or lateral variability that is comparable with the surface roughness of the underlying polycrystalline PbSe layer 42. For some bandpass filter designs, such as narrowband interference filters, such roughness or lateral variability disrupts the “ideal” filter design in which perfectly planar layers of the multilayer stack cooperatively generate constructive and destructive interferences that define the optical pass band. One way to improve the situation is to employ mechanical or chemomechanical polishing of the surface of the PbSe layer 42 prior to deposition of the bandpass filter layer 50 in the embodiment of FIG. 2.

With reference to FIGS. 3 and 4, alternative embodiment integrated devices 30A, 30B are presented. In FIGS. 3 and 4, components corresponding to components of the integrated device 30 of FIG. 2 (namely the substrate 40, PbSe layer 42, electrodes 44, and bandpass filter layer 50) are labeled with corresponding reference numbers. Either the integrated device 30A of FIG. 3 or the integrated device 30B of FIG. 4 may be substituted for the integrated device 30 of FIG. 1, with the light L from the light source 28 oriented as indicated in respective FIGS. 3 and 4. In particular, both the integrated devices 30A, 30B have backside-illuminated configurations in which the light L impinges on the side of the substrate 40 opposite from the side on which the PbSe layer 42 is disposed. The alternative integrated device embodiments 30A, 30B of respective FIGS. 3 and 4 are described in turn below.

With particular reference to FIG. 3, to ensure the light L passes through the bandpass filter layer 50 to reach the PbSe layer 42, in the configuration of FIG. 3 the bandpass filter layer 50 is disposed on the side of the substrate 40 opposite the side on which the PbSe layer 42 is disposed. That is, the bandpass filter layer 50 is disposed on the side on which the light L first impinges. Said another way, the integrated device 30A of FIG. 3 differs from the integrated device 30 of FIG. 2 in that the bandpass filter layer 50 is moved from being deposited on top of the PbSe layer 42 to being deposited on the side of the substrate 40 opposite the side on which the PbSe layer 42 is deposited. The arrangement of FIG. 3 has a potential advantage over the arrangement of FIG. 2 in that the bandpass filter layer 50 in FIG. 3 is deposited onto the (backside) surface of the substrate 40, which is expected to be relatively smooth, rather than being deposited onto the possibly rougher surface of the polycrystalline PbSe layer 42. Thus, the integrated device embodiment 30A may be preferable from a fabrication standpoint in the case of the bandpass filter layer 50 being designed as a multilayer stack with precise thin layer thicknesses defining a narrow pass band. The integrated device 30A also presents an alternative solution to the problem of high reflection from the bare PbSe surface. In the embodiment 30A of FIG. 3, the light L passes through the substrate 40 into the PbSe layer 42, so that the refractive index step encountered is between the refractive index of the substrate 40 and the refractive index of the PbSe layer 42. So long as the substrate 40 has a refractive index that is intermediate between that of air (n=1.0) and PbSe (n=4.9) this configuration should reduce reflection loss. A further design constraint for the integrated device 30A of FIG. 3 is that the substrate 40 should be transparent for the pass band of the bandpass filter layer 50, or in some embodiments at least should be transparent for infrared light at 4.26 micron. By way of some non-limiting examples, in some embodiments of the integrated device 30A the substrate which is transparent for infrared light at 4.26 micron comprises silicon, germanium, zinc selenide, sapphire, titanium oxide, cryolite, magnesium fluoride, zirconia, zinc sulfide, lead fluoride, cadmium fluoride, cadmium sulfide, zinc oxide, tantalum oxide, niobium oxide, antimony fluoride or zerodur.

With particular reference to FIG. 4, in the integrated device 30B the bandpass filter layer 50 is on the same side of the substrate 40 as the PbSe layer 42 (same as in the embodiment of FIG. 2), but in the integrated device 30B of FIG. 4 the bandpass filter 50 is disposed between the substrate 40 and the PbSe layer 42. This arrangement again ensures that the (backside-impinging) light L passes through the bandpass filter layer 50 to reach the PbSe layer 42, so that only the portion of light L that lies in the pass band of the bandpass filter layer 50 reaches the PbSe layer. As with the embodiment of FIG. 3, the embodiment of FIG. 4 has the potential advantage over the arrangement of FIG. 2 that the bandpass filter layer 50 is deposited onto the (frontside) surface of the substrate 40, which is expected to be relatively smooth, rather than being deposited onto the possibly rougher surface of the polycrystalline PbSe layer 42. Thus again, the integrated device embodiment 30B may be preferable from a fabrication standpoint in the case of the bandpass filter layer 50 being designed as a multilayer stack with precise thin layer thicknesses defining a narrow pass band. In the integrated device 30B, the problem of high reflection from the bare PbSe surface is addressed by the light L encountering the smaller refractive index step between the bandpass filter layer 50 and the PbSe layer 42 and/or the ensuing destructive interference effect. So long as the material of the bandpass filter 50 has refractive index that is intermediate between that of air (n=1.0) and PbSe (n=4.9) this configuration should reduce reflection loss.

Although reflection loss at the interface to the PbSe layer 42 is addressed in the integrated device 30B of FIG. 4 as just described, there is potential for reflection loss at the “backside” of the substrate 40, that is, at the side upon which the light L impinges. To reduce this reflection loss, an anti-reflective (AR) coating layer 56 is optionally disposed on the “backside” of the substrate 40, i.e. on the side opposite from the side of the substrate 40 on which the PbSe 42 and bandpass filter layer 50 are disposed. The AR coating layer 56 is suitably any material with refractive index intermediate between that of air (n=1.0) and the refractive index of the substrate 40. Alternatively, the AR coating layer 56 may be a multi-layer stack defining an interference filter having a pass band aligning (at least approximately) with that of the bandpass filter layer 50.

As with the embodiment of FIG. 3, a further design constraint for the integrated device 30B of FIG. 4 is that the substrate 40 should be transparent for the pass band of the bandpass filter layer 50, or in some embodiments at least should be transparent for infrared light at 4.26 micron. By way of some non-limiting examples, in some embodiments of the integrated device 30B the substrate which is transparent for infrared light at 4.26 micron comprises silicon, germanium, zinc selenide, sapphire, or zerodur.

In the embodiment 30B of FIG. 4, the PbSe layer 42 is deposited onto the bandpass filter layer 50. This requires that the bandpass filter layer 50 be impervious to the wet chemical bath used in the chemical bath deposition (CBD) of the PbSe layer 42, or alternatively requires that the bandpass filter layer 50 be impervious to the gas-phase chemistry if vapor phase deposition (VPD) is used to deposit the PbSe layer 42. If the materials of the bandpass filter layer 50 are not capable of withstanding this wet or vapor phase chemistry, then it is contemplated to deposit a thin passivation layer on top of the bandpass filter layer 50 prior to CBD or VPD deposition of the PbSe layer 42.

In the embodiment 30A of FIG. 3, the PbSe layer 42 may be deposited either before or after deposition of the bandpass filter layer 50. If the PbSe layer 42 is deposited after the bandpass filter layer 50, then the bandpass filter layer 50 should either be impervious to the CBD or VPD deposition chemistry, or the bandpass filter layer 50 should be protected by a permanent thin passivation layer or a temporary protective mask layer (e.g. a photoresist layer) that can be removed after deposition of the PbSe layer 42.

In the illustrative embodiments, PbSe is used as the infrared light absorbing layer 42. More generally, the PbSe layer is contemplated to be replaced by another infrared light absorbing layer such as a mercury cadmium telluride layer or an indium antimonide layer. That is, in any of the illustrative embodiments of the integrated component 30, 30A, 30B, the illustrative PbSe layer 42 is contemplated to be replaced by a mercury cadmium telluride layer, an indium antimonide layer, or other infrared light absorbing layer that exhibits photoconductivity in response to illumination by infrared light in a spectral range encompassing the 4.26 micron absorption line for CO₂.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A capnometer comprising:
an integrated device comprising a substrate, an infrared light absorbing layer disposed on the substrate, and a bandpass filter layer disposed on the substrate;
a light source arranged to emit light (L) that passes through the bandpass filter layer of the integrated device to reach the infrared light absorbing layer of the integrated device; and
electronics connected with the infrared light absorbing layer of the integrated device to measure a photoconductivity signal of the infrared light absorbing layer, the electronics including signal processing circuitry to convert the photoconductivity signal to a carbon dioxide partial pressure or concentration value.

2. (canceled)

3. The capnometer of claim 1 wherein the bandpass filter layer and the infrared light absorbing layer of the integrated device are disposed on a same side of the substrate with the bandpass filter layer disposed between the substrate and the infrared light absorbing layer, and the substrate of the integrated device is transparent for the light (L) emitted by the light source at least over the pass band of the bandpass filter layer of the integrated device.

4. The capnometer of claim 3 wherein the integrated device further comprises:
an anti-reflective (AR) coating layer disposed on a side of the substrate opposite from the side on which are disposed the infrared light absorbing layer and the bandpass filter layer.

5. (canceled)

6. The capnometer of claim 1 wherein the pass band of the bandpass filter layer of the integrated device encompasses 4.26 micron.

7. The capnometer of claim 1 wherein the electronics are connected with the infrared light absorbing layer of the integrated device to measure the photoconductivity signal of the infrared light absorbing layer consisting of one of resistance, current, and voltage across the infrared light absorbing layer.

8. The capnometer of claim 1 further comprising: a temperature sensor thermally connected with an integrated device; wherein the electronics are connected with the temperature sensor to monitor the temperature of the integrated device.

9. The capnometer of claim 1 further comprising: a carbon dioxide measurement cell including a flow path (F) through which respired gas flows; wherein the light source is arranged to emit the light (L) to pass through the flow path (F) of the CO₂ measurement cell before reaching the bandpass filter layer of the integrated device.

10. The capnometer of claim 1 wherein the infrared light absorbing layer of the integrated device is a lead selenide (PbSe) layer.

11. An infrared light detector comprising:
a substrate;
a lead selenide (PbSe) layer disposed on the substrate;
electrodes disposed on the substrate and electrically connected with the PbSe layer; and
a bandpass filter layer disposed on the substrate, the bandpass filter layer having a pass band encompassing 4.26 micron.

12. (canceled)

13. The infrared light detector of claim 11 wherein the bandpass filter layer and the PbSe layer are disposed on a same side of the substrate with the bandpass filter layer disposed between the substrate and the PbSe layer, and the substrate is transparent for infrared light at 4.26 micron.

14-16. (canceled)

17. The infrared light detector of claim 1 wherein the bandpass filter layer comprises a multi-layer stack defining an interference filter.

18. The infrared light detector of claim 13 wherein the substrate which is transparent for infrared light at 4.26 micron comprises silicon, germanium, zinc selenide, sapphire, titanium oxide, cryolite, magnesium fluoride, zirconia, zinc sulfide, lead fluoride, cadmium fluoride, cadmium sulfide, zinc oxide, tantalum oxide, niobium oxide, antimony fluoride or zerodur.

19. The infrared light detector of claim 11 wherein the PbSe layer has a thickness between 0.5 micron and 2.5 micron inclusive.

20. The infrared light detector of claim 11 further comprising:
a light source arranged to emit light (L) that passes through the bandpass filter layer to reach the PbSe layer; and
electronics connected with the electrodes to measure a photoconductivity signal of the PbSe layer.

21. The infrared light detector of claim 20 wherein the photoconductivity signal is one of resistance, current, and voltage across the PbSe layer.

22. The infrared light detector of claim 20 further comprising:
a temperature sensor thermally connected with an integrated filter/detector device comprising the substrate, the PbSe layer disposed on the substrate, the electrodes disposed on the substrate, and the bandpass filter layer disposed on the substrate;
wherein the electronics are connected with the temperature sensor to monitor the temperature of the integrated filter/detector device.

23. The infrared light detector of claim 20 wherein the electronics include signal processing circuitry to convert the photoconductivity signal to a carbon dioxide partial pressure or concentration value.
